(19) United States
(12) Patent Application Publication
(10) Pub. No.: US 2006/0058745 A1
(43) Pub. Date: Mar. 16, 2006

(54) Smart disposable diaper with sanitary transmitter

(76) Inventor: Harold George Pires, Easton, PA (US)

Correspondence Address: Benita J. Rohm;Rohm & Monsanto, PLC, 12 Rathbone Place, Grosse Pointe, MI, 48230, US

(21) Appl. No.: 11/223,895

(22) Filed: Sep. 9, 2005

Related U.S. Application Data
(63) Continuation-in-part of application No. 10/997,825, filed on Nov. 23, 2004.
Application No. 10/997,825 is a continuation-in-part of application No. 10/723,604, filed on Nov. 25, 2003.
(60) Provisional application No. 60/499,191, filed on Sep. 2, 2003.

(30) Foreign Application Priority Data
Aug. 31, 2004 (WO) PCT/US04/28464

(51) Int. Cl. A61F 13/20

(52) U.S. Cl. 604/361

(57) ABSTRACT

A diaper product is formed by the steps of first installing a sensor element onto a first layer of the plurality of layers of material; second installing a layer of absorbent material onto the first layer of the plurality of layers material; and forming the combined first layer of the plurality of layers of material with the sensor element installed thereon and the layer of absorbent material into a disposable diaper product. Electrical contacts communicate with the sensor element. A transmitter is installed within a transmitter housing that has first and second portions that are pivotally joined to one another, and is arranged to transmit a signal responsive to the sensor element and to a duration of a predetermined period of time that corresponds to the duration of a selectable condition of the diaper. A receiver responds to predetermined transmitters, each transmitter being associated with a respective patient.

### RELATIONSHIP TO OTHER APPLICATIONS

This application is a continuation-in-part patent application of U.S. patent application Ser. No. 10/997,825, filed Nov. 25, 2004, which is a continuation-in-part of U.S. patent application Ser. No. 10/723,604, filed Nov. 25, 2003, which claims the benefit of Provisional U.S. Patent Application Ser. No. 60/499,191, filed on Sep. 2, 2003. The disclosures of all of these applications are incorporated herein by reference.

### BACKGROUND OF THE INVENTION

1. Field of the Invention

This invention relates generally to systems for determining a wet environmental condition from a remote location, and more particularly, to a simple and economical system for announcing a wet diaper condition at a remote monitor.

2. Description of the Related Art

There has long been a need for the detection of soiled diapers and remote notification of a care giver. This is important in the case of babies who will develop diaper rash when left for long periods of time in a soiled diaper, and even more important in the case of incontinent adults who are unable to communicate with attendants. Adults are also susceptible to diaper rash, and in adults this is a far more serious condition that is more difficult to treat and cure than for babies. In some adult care institutions, diapers are termed “briefs.” However, the term “diaper” will herein be applied to encompass all these devices.

In baby day care centers, diapers are usually changed at timed intervals, which does not solve the diaper rash problem because a baby can soil his or her diaper soon after the diaper change. The baby must then remain in a soiled diaper until the next scheduled change. Of course, this problematical situation also applies to incontinent adults. In addition, there are a many adults who are ambulatory, but have lost bladder control and sensitivity in the groin area. Such adults realize they are soiled only after the diaper leaks, causing embarrassment.

One known form of incontinence protection is an absorbent rectangular sheet called an “underpad” that is placed under the patient and performs substantially the same function as a diaper. This known device suffers from the same shortcomings of diapers in that the care giver is not notified when the underpad is wetted or soiled.

Several attempts have been made in the prior art to provide remote notification capability, but all have failed to be accepted by the general public. To be successful with consumers and manufacturers, the entire system has to meet at least the following basic criteria:

(a) be very inexpensive;

(b) be easy for the attendant to use;

(c) have a transmitter that is easy to clean;

(d) have a useful range of operation;

(e) place no restriction on the mobility of either the diaper wearer or the attendant; and

(f) be easily manufactured without requiring significant retooling of conventional diaper manufacturing equipment.

The products in this field have been “after market” devices for diapers that fail meet these requirements. These after market products are required to be installed during each diaper change, and therefore place a significant burden on the attendant care giver. More specifically, the care giver is required to perform additional operations during the diaper change, some of which are difficult, and these are multiplied by the fact that diapers are changed eight to ten times a day. Therefore, even small additional burdens are multiplied. One known method of providing care to the incontinent is to change the diapers at predetermined intervals, such as every two hours. Often times, a dry diaper is changed, and the patient wets or soils the new diaper shortly after the change. The diaper change may itself stimulate the wetting or soiling, and the patient will therefore wait almost two hours in a wet or soiled diaper before the next scheduled diaper change. As this pattern is repeated, the patient's risk of developing a rash or contracting infection is increased.

In addition to the foregoing, as a health care product, the importance of having a transmitter that is easy to clean cannot be over emphasized. This aspect of control over the spread of infection and disease has been missed by the known after market products.

It is, therefore, an object of this invention to provide a simple and economical bodily fluid containment arrangement and notification system that reduces the incidence of rash.

It is another object of this invention to provide a simple and economical bodily fluid containment arrangement and notification system that reduces the spread of infection and disease.

It is also an object of this invention to provide a simple and economical bodily fluid containment arrangement and notification system that is easy to clean and maintain.

It is a further object of this invention to provide a simple and economical diaper arrangement and notification system that reduces the incidence of diaper rash.

It is additionally an object of this invention to provide a simple and economical diaper arrangement and notification system that provides indication of the period of time that a diaper has been wetted or soiled.

It is yet a further object of this invention to provide a simple and economical diaper arrangement and notification system that provides indication of the period of time that a diaper has remained dry or unsoiled.

It is also another object of this invention to provide a simple and economical diaper arrangement and notification system that provides reliable electrical interconnection between a transmitter and a diaper product.

It is yet an additional object of this invention to provide a simple and economical diaper arrangement and notification system that is responsive to predetermined coded signals associated with predetermined patients or babies.

It is still another object of this invention to provide a simple and economical diaper arrangement and notification system that can easily be programmed to be responsive only to predetermined coded signals.

### SUMMARY OF THE INVENTION

The foregoing and other objects are achieved by this invention, which provides a diaper product having a plurality of layers of material for use by a living being. The diaper product is, in accordance with the invention, formed by the steps of:

first installing a sensor element onto a first layer of the plurality of layers of material;

second installing a layer of absorbent material onto the first layer of the plurality of layers material; and

forming the combined first layer of the plurality of layers of material with the sensor element installed thereon and the layer of absorbent material into a disposable diaper product having a plurality of layers of material.

There is additionally provided the step of first attaching a first electrical contact to communicate electrically with the sensor element. In embodiments where the sensor element includes first and second conductive elements, the step of first attaching is performed to attach the first electrical contact to communicate electrically with the first conductive element of the sensor element, and there is provided the further step of second attaching a second electrical contact to communicate electrically with the second conductive element of the sensor element. The sensor element has a first electrical characteristic when the first layer of the plurality of layers of material is dry, and a second electrical characteristic when the first layer of the plurality of layers of material is wet.

In a preferred embodiment, the first layer of material is formed from a continuous web of first layer material, and the step of first installing a sensor element onto a first layer of material includes the step of depositing a stripe of conductive material on the continuous web of first layer material. The stripe of conductive material is a hot melt conductive material in some embodiments, or may be a printed conductive material.

In a specific illustrative embodiment of the invention, the first layer of material is an outer layer of the diaper product. Alternatively, the first layer of material is a substrate for forming a wetness sensor with the stripe of conductive material deposited thereon.

In accordance with the invention, there is further provided a transmitter housing arranged to communicate electrically with the electrical contact. The transmitter housing includes first and second portions that are pivotally joined to one another, the first and second portions, when the transmitter housing is installed, being configured to overlie the first layer of material. A resilient clamping element urges the first and second portions of the transmitter housing toward a closed position. The resilient clamping element includes, in a specific illustrative embodiment of the invention, a magnetic element.

Further in accordance with the invention there is provided a transmitter installed within the transmitter housing. The transmitter is arranged to transmit a signal responsive to the sensor element. In a further embodiment, the transmitter transmits a further signal that is responsive to the sensor element and to a duration of a predetermined period of time. The period of time corresponds to the duration of the wet condition of the diaper, or to the duration of a dry condition of the diaper, as such would indicate a serious medical problem with the patient.

A high degree of hygiene is achieved by encasing the transmitter in a waterproof material. In a preferred embodiment, the waterproof material constitutes the transmitter housing and extends to form a resilient hinge that pivotally joins the first and second portions of the transmitter housing to one another. The resilient hinge is substantially free of crevices that can harbor bacteria.

Prior to performing the step of forming, there is provided the further step of third installing an impermeable shield that precludes contact between the sensor element and the skin of the living being. This serves to protect the patient from contact with the conductive material, notwithstanding that there are no dangerous voltages present in the sensor. In some embodiments, the layer of absorbent material is configured to preclude contact between the sensor element and the skin of the living being.

In accordance with a diaper wetness monitoring system aspect of the invention, there is provided a transmitter arrangement having a transmitter housing with first and second portions that are pivotally joined to one another for straddling an edge of a diaper. A resilient clamping element urges the first and second portions of the transmitter housing toward a closed position with the diaper interposed therebetween. A transmitter is installed within the transmitter housing, the transmitter being electrically coupled to an electrical contact installed on a selectable one of the first and second portions. The electrical contact is arranged to communicate electrically with a corresponding electrical contact of the diaper in response to the urging by the resilient clamping element.

In one embodiment of the diaper monitoring system the resilient clamping element includes a magnetic element. The transmitter is installed within the first portion of the transmitter housing, and the electrical contact is disposed on the exterior of the first portion of the transmitter housing so as to be intermediate of the first and second portions when in the closed position. There is further provided an electrical interconnection arrangement between the contact and the transmitter.

In a preferred embodiment of the diaper monitoring system, the transmitter is encased in a waterproof containment arrangement that extends outward of the transmitter housing to form a hinge that joins the first and second portions of the transmitter pivotally to one another. The waterproof containment arrangement is devoid of crevices that can harbor bacterial growth. It is preferred that the waterproof containment arrangement encase the transmitter housing.

In a preferred embodiment of the invention, the transmitter is arranged to transmit a signal containing data uniquely associated with the transmitter. There is further provided a receiver for receiving the signal containing the data uniquely associated with the transmitter. The receiver is configured to learn the data uniquely associated with, and received from, the transmitter.

In a specific illustrative embodiment of the invention, the transmitter has active and quiescent modes of operation, and there is further provided a timer for timing the duration of a mode of the transmitter. In a bodily fluid containment environment, it is desirable to know the duration of a period of wetness of the absorbent layer, as well as the duration of a period of dryness.

In accordance with a further aspect of the invention, there is provided a bodily fluid containment product for a living being, the bodily fluid containment product being formed in plural stages of manufacture. The bodily fluid containment product is provided with an absorptive inner layer for communicating with the bodily fluid and an impervious outer layer for containing the bodily fluid. A wetness sensor is disposed intermediate of the absorptive inner layer and the impervious outer layer, the absorptive inner layer, the impervious outer layer, and the wetness sensor being combined concurrently during manufacture. Additionally, an electrical contact is coupled electrically to the wetness sensor.

The conditions of being very inexpensive and easy for the attendant to use are achieved by including the sensor as an integral part of the diaper. Preferably, such inclusion is effected as a process step during the manufacture of the diaper. The invention tackles the problem at the root by making the diaper itself “smart.” Thus, when the user purchases the diaper, the wetness sensor is already incorporated into the product.

Diapers are manufactured by very expensive fully automatic machines that run at approximately 900 feet per minute. The present technology is implemented by making relatively minor modifications to the known machines, and therefore is commercially practical and economical to implement.

Diapers of the disposable type consist of several layers and sections. There is an outside layer that is formed plastic, but is made to look and feel like cloth. This is termed a “non woven” material. The next layer is formed of polypropylene or polyethylene and is impervious to moisture. Then there is provided a layer of absorbent flock material. Above that there is typically provided another layer porous material that is soft and is the layer that touches the skin of the patient. These layers start off during the manufacturing process as large rolls that are fed into the diaper making machines. The machine processes the rolls by cutting, shaping, and processing the pieces, including the spraying of a glue that holds the layers together in order to create the final diaper product. The combined two outer layers are herein referred to as the “outer layer,” and the flock material together with the porous inner layer are referred to as the “absorbent layer.” Underpads have basically the same structure.

The system has three basic components. One component is the smart disposable diaper containing the wetness sensor and the electrical connections necessary to communicate directly with an external transmitter. Another component is the transmitter mounted within a housing that is easily transportable from a wet diaper to a clean one, and the third component is a receiver that is designed to respond selectively to individual diapers. In accordance with the invention, several types of receivers are available to customers depending on the application. Nevertheless, all of the receivers are designed to learn individual diaper codes and thereafter respond only to the learned code.

The sensor in the diaper is, in a practicable embodiment of the invention, configured as two stripes of electrically conductive material placed either directly on the inner surface of the outer layer of the diaper or on a separate narrow strip of flexible plastic that is then attached to the inner surface of the outer layer. These stripes are situated between the absorbent layer and the outer layer and therefore do not contact directly the skin of the wearer. When the diaper wearer urinates, urine soaks into the absorbent layer and shortly thereafter contacts the stripes, thereby reducing the impedance between them. Defecation is accompanied by urination, so the wetness sensor detects both types of excretions. The absorbent layer prevents small droplets of urine or sweat from reaching the sensor, thereby eliminating false triggering of the system.

The stripes are installed longitudinally from the crotch area extending all the way in the front to the waistband of the diaper. The section of the stripes near the crotch area function as a wetness sensor. The section of the stripes near the waistband function as contacts for effecting the connection to an external transmitter. The portion of the stripes located between the crotch and the contacts constitute the electrical connection between the wetness sensor and the contacts.

Transmitters preferably are mounted in a completely sealed waterproof housing that is equipped with a hinged flap. Flexible magnets are placed both on the flap and the side of the housing facing the flap. The housing assembly is mounted on the diaper so as to straddle the waistband, with the flap on one side of the diaper material and the transmitter on the other side. Magnetic attraction between the flap and the body of the transmitter housing securely holds the transmitter housing in place. The transmitters in a multi-transmitter system are individually incrementally coded during manufacture with a bit stream long enough not to require repeating over decades of years.

In one embodiment, the transmitters use radio frequency identification (“RFID”) technology. Also, the transmitters in certain embodiments each contain a non-replaceable battery having an operational life that may exceed two years.

There are contemplated within the invention three forms of receivers. Parents will carry portable, belt mounted beeper receivers designed to respond to a few, illustratively three, individual transmitters because it is not uncommon to have more than one baby in diapers at the same time or twins and a single baby. Baby day care and adult care institutions will use multiple unit receivers that can handle a number of diaper users. Baby care receivers can learn the same codes as the parent's receivers, and therefore the transmitter that activates the baby care receiver during the day will activate the parent's receiver in the evening. Hospitals and large patient care establishments require receivers with more elaborate features.

Diaper manufacturing machines are available from several companies. The choice between the two embodiments described above is determined by the ease and cost of modifications required for any individual machine. Diaper making machines are frequently modified to accommodate improvements to diapers, and the modifications required by this invention are within the scope of modifications that typically are made to such machines.

The smart diapers of the present invention offer several major advantages for wetness sensing, both from a manufacturer's as well as a user's point of view. These diapers have significant commercial value. The other known efforts at remote wetness sensing for diapers have used after market products, and therefore none has been successful at large scale production. More specifically, they fail to meet the requirements stated earlier.

From a manufacturing point of view, it is cheap and easy for a modified diaper making machine to manufacture our diapers, because they do not require difficult operations such as the addition of holes or metallic contacts. Our method of using conductive strips for the purposes of making contact with an external transmitter, including the method of ensuring registration between contacts and conductive strips, is very effective. It is unique and a very inexpensive and uses no holes or complex registration methods. Our method of registration is simple, using wide contacts on the transmitter and an outline marking the outside of the diaper to show the location of the transmitter. It is the only practical method for the highly automated diaper making machines. Our invention takes advantage of the absorbent layer to avoid false triggering, by placing the conductive stripes (for wetness sensing) on the side of the absorbent layer that is away from the skin. This a big advantage over after market products, which by their very nature cannot go on that side of the absorbent layer and have to go on the skin side of the absorbent layer.

From a user's point of view, the diaper of the present invention advantageously presents no additional burden to the user, since both the sensor and contacts are already built-in. The present diapers do not produce false indications of wetness because they are not susceptible to sweat or droplets of urine. The conductive stripes do not contact with the skin of the wearer. The diapers are inexpensive and contain no wires, Wires are a psychological deterrent to users. In addition, transmitter, particularly when it is housed within an encasement of waterproof material, has no crevices and is easy to keep clean.

### BRIEF DESCRIPTION OF THE DRAWING

Comprehension of the invention is facilitated by reading the following detailed description, in conjunction with the annexed drawing, in which:

FIG. 1 is a schematic representation of an inside view of an outer layer of an open diaper, showing two conductive stripes;

FIG. 2 is a schematic representation of an absorbent layer;

FIG. 3 is a schematic representation of an assembly containing a wetness sensor;

FIG. 4 is schematic representation of a flexible strip with conductive stripes placed near the outer edges of the strip;

FIG. 5 is a schematic representation of an assembly containing the wetness sensor formed by the flexible strip of FIG. 4;

FIG. 6 is a schematic representation of a transmitter housing and flap that is to be used with the housing inside of the diaper and the flap on the outside;

FIG. 7 is a schematic representation of a transmitter housing and flap that is to be used with the flap inside of the diaper and the housing on the outside;

FIG. 8 is a schematic representation of the surface of the flap of FIG. 7 that faces the housing;

FIG. 9(a) is a schematic representation of the surface of the flap and FIG. 9(b) is a representation of a side view the flap of FIG. 9(a);

FIG. 10 is a schematic representation of a beeper receiver that can be carried on the belt of a care giver;

FIG. 11 is a schematic representation of a transmitter installed on the waistband of the diaper.

FIG. 12 is a schematic representation of the transmitter housing with the flap open at 180°.

### DETAILED DESCRIPTION

FIGS. 1, 3, and 5 illustrate schematic representations of a diaper product embodiment of the invention in an open condition and in sequential states of completion of manufacture. Correspondingly analogous elements of structure are similarly designated in the various figures.

Leg openings 3, are formed in the crotch area of the diaper to accommodate the legs (not shown) of a wearer (not shown). Bottom edge 15 of the diaper arrangement represented in FIGS. 1-5 is folded forwards and up (not shown in this figure), and constitutes the front of the diaper. Top edge 16 corresponds to the back of the diaper. The edges 15 and 16 are configured to be at waist level (not shown) when the diaper is installed on the wearer, and therefore form part of the waistband (not shown) of the diaper.

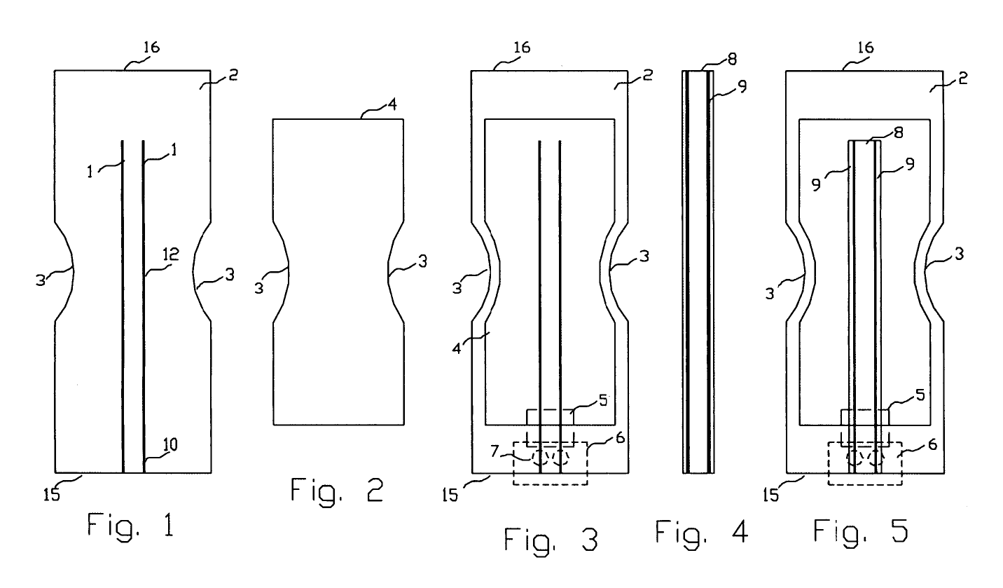

One embodiment of the sensor portion of the invention is represented in FIGS. 1 and 3. FIG. 1 shows outer layer 2 having deposited thereon a pair of conductive stripes 1. The conductive stripes are installed on outer layer 2 either by a hot melt gun (not shown) or a printing system (not shown) installed on a diaper making machine (not shown). Hot melt guns can be started and stopped very quickly and can be timed to create the stripes in the diaper making machine. Printing of the stripe is achieved with a printing and registration process (not shown). The section of stripes 12 near the crotch area (not specifically designated) forms the wetness sensing portion. Portion 10 of the stripes located near the waistband (edge 15) forms electrical contacts for an external transmitter (not shown in this figure). FIG. 2 shows an absorbent layer 4. FIG. 3 shows the relative positions of conductive stripes 1, absorbent layer 4, and a transmitter 6 (shown in outline form) placed on the diaper as shown, and discussed below in relation to, in FIG. 11. Transmitter 6 is typically not manufactured simultaneously with the diaper product. In a baby diaper embodiment of the invention, outer layer 2 is approximately seven inches wide by eighteen inches long.

Two transmitter contacts 7 are shown to communicate with conductive stripes 1. The conductive stripes do not extend beyond the absorbent layer at the back (edge 16) of the diaper. In the front of the diaper (edge 15) conductive stripes 1 extend beyond the edge of the absorbent layer so as to reach contacts 7 of transmitter 6. The section of conductive stripes 1 between the end of the absorbent layer and the transmitter is exposed and capable of touching the skin (not shown) of the wearer. However, in order to prevent such skin contact, a small rectangular impermeable shield 5 is, in this specific illustrative embodiment of the invention, glued directly onto outer layer 2 under absorbent layer 4 and the location of transmitter 6.

A further embodiment of the invention is depicted in FIGS. 4 and 5. FIG. 4 shows two conductive stripes 9 deposited near the edges, and in this embodiment on the same side, of a thin film 8. In a specific illustrative embodiment of the invention, thin film 8 is formed of polyethylene or polypropylene about half an inch wide. In the practice of the invention, thin film 8 and conductive stripes 9 thereon are manufactured in a printing machine (not shown) external to the diaper-manufacturing machine (not shown). Thin film 8 is then formed into a roll (not shown) that is then fed to the diaper making machine. The rolled thin film is treated as another layer by the diaper machine similar to the other rolls (not shown) that are used to form the other diaper layers.

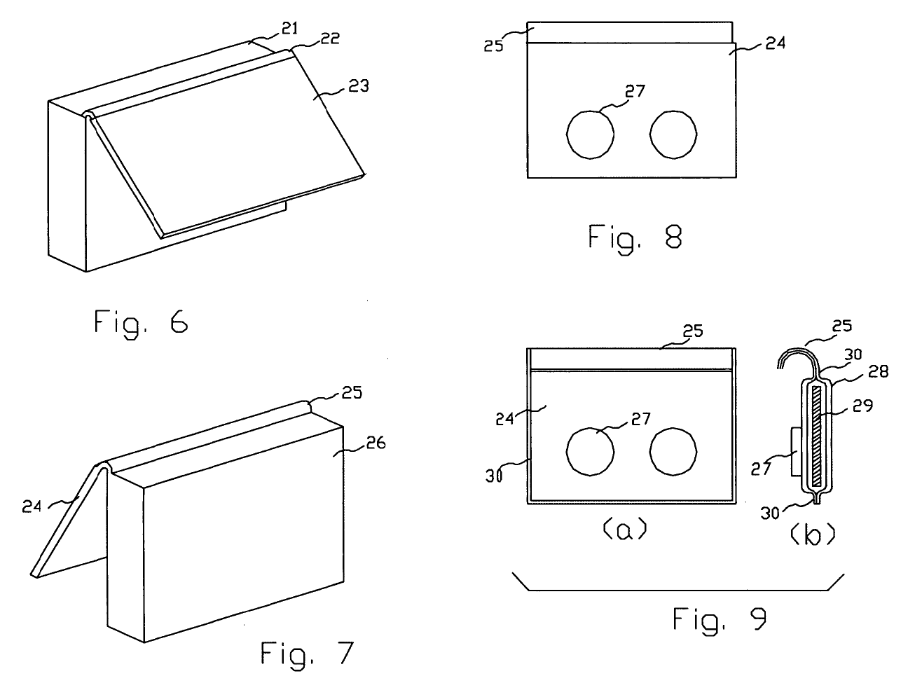

In accordance with the invention, transmitter 6 senses the impedance between the two conductive stripes in the diaper. Two embodiments of such a system are shown in FIGS. 6 and 7. In FIG. 6, the transmitter (not shown in this figure) is installed in a housing 21. In FIG. 7 the transmitter (not shown in this figure) is installed in a housing 26. In these embodiments, respective flaps 23 and 24, and the respective surfaces of housings 21 and 26 that face toward the respective flaps have incorporated therein flexible magnetic strips, as will be discussed below. In use, the diaper material that contains the conductive stripes is placed between the flaps and the respective transmitter housings. The magnetic strips attract the flap to the housing and the pressure of the attraction performs two functions. First, it holds the housing firmly in place and helps contacts 27 (FIGS. 8 and 9) to make a good electrical connection with the conductive stripes. In this specific illustrative embodiment of the invention, contacts 27 are about 10 thousandths of an inch thick and are formed to have a watertight seal around their edges.

In the embodiment of FIG. 6, contacts 27 (not shown) are installed on the side of transmitter housing 21 that faces flap 23. An electrical communication (not shown) is made with the transmitter circuitry (not shown) that is contained within the housing. When installed on the diaper, housing 21 is positioned inside the diaper (not shown in this figure) whereby and contacts 27 communicate with the conductive stripes (not shown in this figure) in the diaper.

In the embodiment of FIG. 7, contacts 27 (not shown) are installed on the inside surface of flap 24. An electrical connection (not shown) is formed between contacts 27 and the electronic transmitter circuitry (not shown) that is contained within housing 26. In this embodiment of the invention, the electrical connection is established using conductors (not shown) that are routed through hinge 25.

An advantageous aspect of the embodiment depicted in FIG. 6 is that the electrical contacts can be placed directly on the housing that contains the electronics, and there is accordingly no need to route conductors along the hinge to the flap. On the other hand, the embodiment of FIG. 7 with the contacts on the flap affords the advantage that the flap is disposed inside the diaper, the flap being thinner than the housing. Some customers may prefer that configuration. Current technology enables the transmitter to be made very small, and in some embodiments, the thickness of the transmitter housing may be on the order of one third of an inch. This permits the housing to be placed within the diaper without causing discomfort. From the standpoint of patient comfort, there is little difference between the housing being on the inside or outside, since the combined thickness of the flap and the housing will remain the same, and that is what would make a significant effect on patient comfort. Hinge 35 is located over the waistband of the diaper in either configuration.

The transmitter assembly of the present invention affords several advantages over prior art arrangements. It does not require a special holder, it is easy to install on a smart diaper, and is easy to disconnect. The use of flexible magnetic strips to hold the transmitter in place solves several problems. It ensures a good electrical connection with both contacts, whereas if a spring were to be used for the purpose of closing the flap, the quality of the electrical connection would depend upon the hinge making the flap perfectly parallel to the surface of the housing. More specifically, any unevenness would make one contact touch its respective stripe before the other, thereafter preventing the flap from moving further and preventing communication between the second stripe and the second contact. In order to accommodate manufacturing tolerances and thereby correct this pronblem, each contact would have to be made resilient, resulting in increased manufacturing cost. The flexibility of the magnetic flap ensures a good solid pressure for both contacts, regardless of the extent of unevenness of the hinge or diaper. Magnets also make the housing assembly easy to keep clean. The magnetic flap can be opened 180°, substantially as a flat surface as shown in FIG. 12.

FIG. 8 and FIG. 9 are schematic representations of flap 24 of the embodiment of FIG. 7. For the sake of clarity, transmitter housing 26 is not shown. Hinge 25 is shown only to the point where it touches the housing. Thin vinyl film 28 encases flexible magnetic strip 29. The edges of the vinyl 30 are subjected to heat and pressure in this embodiment to achieve a water tight seal. As shown, the vinyl is extended to form hinge 25. The hinge is attached (not shown) to the body of the housing. In one embodiment the vinyl film and edge sealing may be further extended beyond the hinge to cover the entire transmitter housing. In that embodiment, the housing may be eliminated entirely relying on the vinyl sheath to cover and house the electronic components, in which case the vinyl constitutes the transmitter housing. Metal contacts 27 are in the form of special gold plated units similar to thumb tacks. The pins (not shown) of the “thumbtacks” are electrically connected to the transmitter (not shown) located in the transmitter housing by means of conductors (not shown) that are routed through hinge 25 to housing 26.

In the embodiment of FIG. 6, the flap does not contain the contacts, but the construction of the flap is otherwise similar to that shown in FIG. 9(b).

In a preferred embodiment, the transmitter housing also has a magnetic strip encased in a waterproof material, such as vinyl, on the surface facing the flap. In both embodiments, the entire casing of the housing, including the flap containing the magnetic strip, is a sanitary construction with smooth surfaces and without any crevices that could harbor dirt or germs.

When used as herein described, the transmitter will be located near the waistband and will generally not become soiled between diaper changes. However, the transmitter will require cleaning occasionally. The flap is designed to be opened at 180 degrees to a flat configuration as shown in FIG. 12, to facilitate cleaning.

FIG. 11 is a schematic representation that shows a transmitter 37 arranged in place over the waistband of a diaper 36. Hinge 35 lies over the top of the waistband, whereby the first and second portions of the transmitter housing straddle the waistband. Both embodiments, (transmitter housing inside diaper and transmitter housing outside diaper) are schematically represented by this view. To facilitate proper alignment of contacts 27 on transmitter housing and contacts 10 on the diaper, an outline 38 is optionally printed on the outside surface of the waistband.

From a usability standpoint, the present flap arrangement for communicating with the conductive stripes on the diaper is significantly advantageous over a known housing with spring clips. Such known spring clips are not useable with the diapers of the present invention, as they are useable only with cloth diapers. More specifically, the known spring contacts (biased clips) would scratch the conductive stripes as they are sliding over them during insertion. This would damage the stripes and render the electrical connection unreliable. The contacts of the present invention, on the other hand, move substantially perpendicularly (pivotally arcuately) towards the conductive stripes during the process of making electrical contact. There is therefore no sliding or scratching motion that would damage the integrity of the electrical connection. In addition, the flexible magnets in the flap can accommodate the contours of the housing and the diaper material to enable the solid contacts to make good electrical connection without the complex arrangement using registration “prongs” or “contact prongs” as in some prior art arrangements. Such prior art arrangements are expensive to make and install, and have crevices that are difficult to keep clean.

During diaper changes using the present invention, the following procedure is employed: The attendant first removes the transmitter housing from the soiled diaper and attaches it to the correct position on the clean diaper. He or she then removes the soiled diaper, cleans the patient and installs the clean diaper (with the attached transmitter) on the patient. The transmitter assembly will not require cleaning between diaper changes.

The transmitter, not shown, may in some embodiments use radio frequency identification (“RFID”) technology, and includes a circuit (not shown) that senses the impedance presented to contacts 27. When this impedance drops below a predetermined level, the transmitter is triggered to emit a radio frequency signals in short bursts at regular intervals. During manufacture each transmitter is assigned a unique code, using a large number of bits. Repetition of the code will not occur for several decades of years. Receivers are programmed to respond to specific codes. Therefore, when a receiver receives a transmission, it checks the incoming code with its own reference code. If the incoming code matches one of the predetermined internal codes of the receiver, it flashes the appropriate LED and emits an audible tone or an electronic signal or vibrates mechanically or provides some other indication that it has correctly deciphered the transmitted code. Transmitters are housed in a water proof case with a non-replaceable battery (not shown) having an estimated life of about two years. At the end of the battery life, the transmitter will be discarded.

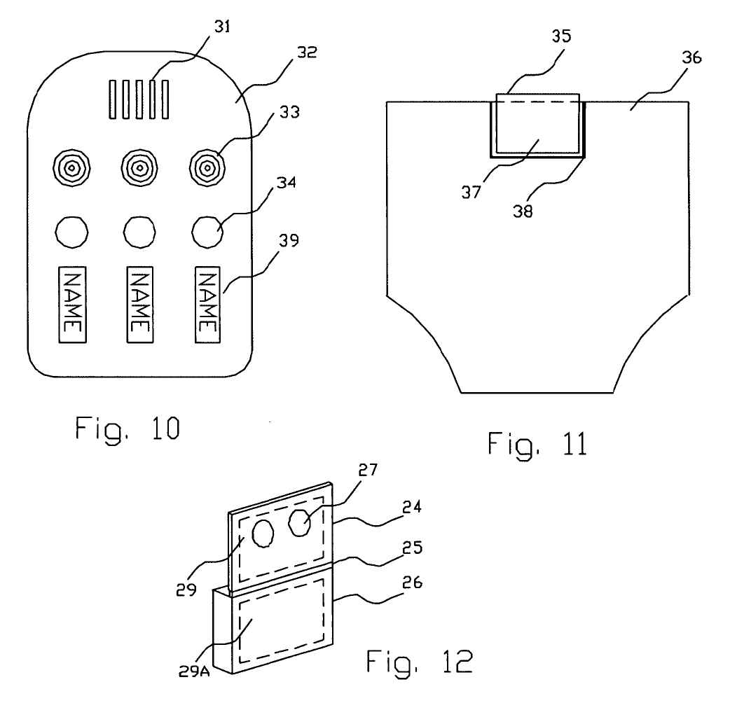

FIG. 10 is a schematic representation of one of the receivers designed to be carried on a belt (not shown) or left on a table top (not shown). The receiver will run on replaceable primary cells (not shown) having a life of about one year. When the battery reaches near the end of its life, the receiver will provide indication in one embodiment with a predetermined beep. The receiver has a sounding device (not shown) that is externally audible through via slots 31. There are additionally provided, three light-emitting diodes (“LEDs”) 33, three associated switches 34, one for each LED, and three name tags 39. The name tags enable the inclusion of handwritten names of the patients or babies being monitored. Whenever the receiver detects a wet diaper, the corresponding LED flashes and an audible alarm is sounded.

The receiver is configured to learn the codes from individual transmitters. To initiate the learning sequence, the user places a wet finger across the transmitter contact and contemporaneously presses one of the learn buttons 34. The corresponding LED 33, flashes to indicate that the learning process is complete. The user releases the learn button and removes the wet finger from the transmitter. To confirm that the learning occurred properly, the user waits a few seconds and again touches the two contacts with a wet finger. The transmitter will issue an audible alarm and the corresponding LED will flash. This unit will learn the signal from three separate transmitters to accommodate the fact that there could be more than one baby in diapers at the same time, which may include a set of twins.

This invention has several advantages over known systems and methods. Since the sensor and the connections to the transmitter are inherent parts of the diaper, instead of accessories thereto, the cost is reduced sufficiently to achieve commercial practicality, as previously noted. In addition, the transmitter connects to the sensor in the diaper without the use of metal contacts or holes in the diaper. The installation of metal contacts and holes is difficult and costly when using diaper making machines of the type that operate at some 900 feet per minute. Moreover, some diaper machines are equipped with metal detectors for identifying broken machine parts, thereby precluding the use of metal parts in the diapers themselves.

Although the invention has been described in terms of specific embodiments and applications, persons skilled in the art may, in light of this teaching, generate additional embodiments without exceeding the scope or departing from the spirit of the claimed invention. Accordingly, it is to be understood that the drawing and description in this disclosure are proffered to facilitate comprehension of the invention, and should not be construed to limit the scope thereof.

What is claimed is:

1. A diaper product having a plurality of layers of material for use by a living being, the diaper product formed by the steps of:
first installing a sensor element onto a first layer of the plurality of layers of material;
second installing a layer of absorbent material onto the first layer of the plurality of layers material; and
forming the combined first layer of the plurality of layers of material with the sensor element installed thereon and the layer of absorbent material into a disposable diaper product having a plurality of layers of material.

2. The diaper product of claim 1, wherein there is provided the step of first attaching a first electrical contact to communicate electrically with the sensor element.

3. The diaper product of claim 2, wherein the sensor element comprises first and second conductive elements, said step of first attaching is performed to attach the first electrical contact to communicate electrically with the first conductive element of the sensor element, and there is provided the further step of second attaching a second electrical contact to communicate electrically with the second conductive element of said sensor element, said sensor element having a first electrical characteristic when the first layer of the plurality of layers of material is dry, and a second electrical characteristic when the first layer of the plurality of layers of material is wet.

4. The diaper product of claim 1, wherein the first layer of material is formed from a continuous web of first layer material, and said step of first installing a sensor element onto a first layer of material comprises the step of depositing a stripe of conductive material on the continuous web of first layer material.

5. The diaper product of claim 4, wherein the stripe of conductive material is a hot melt conductive material.

6. The diaper product of claim 4, wherein the stripe of conductive material is a printed conductive material.

7. The diaper product of claim 4, wherein the first layer of material is an outer layer of the diaper product.

8. The diaper product of claim 4, wherein the first layer of material is a substrate for forming a wetness sensor with the stripe of conductive material deposited thereon.

9. The diaper product of claim 2, wherein there is further provided a transmitter housing arranged to communicate electrically with the electrical contact.

10. The diaper product of claim 9, wherein said transmitter housing is comprised of first and second portions that are pivotally joined to one another, the first and second portions, when the transmitter housing is installed, being configured to overlie the first layer of material.

11. The diaper product of claim 10, wherein there is further provided a resilient clamping element for urging the first and second portions of the transmitter housing toward a closed position.

12. The diaper product of claim 11, wherein said resilient clamping element comprises a magnetic element.

13. The diaper product of claim 10, wherein there is further provided a transmitter installed within the transmitter housing.

14. The diaper product of claim 13, wherein said transmitter is arranged to transmit a signal responsive to said sensor element.

15. The diaper product of claim 13, wherein said transmitter is arranged to transmit a signal responsive to said sensor element and to a duration of a predetermined period of time.

16. The diaper arrangement of claim 13, wherein said transmitter housing is formed of a waterproof material that encases said transmitter.

17. The diaper arrangement of claim 16, wherein the waterproof material extends to form a resilient hinge that pivotally joins the first and second portions of said transmitter housing to one another.

18. The diaper arrangement of claim 17, wherein the resilient hinge is substantially free of crevices that can harbor bacteria.

19. The diaper product of claim 1, wherein, prior to performing said step of forming, there is provided the further step of third installing an impermeable shield that precludes contact between the sensor element and the skin of the living being.

20. The diaper product of claim 1, wherein the layer of absorbent material is configured to preclude contact between the sensor element and the skin of the living being.

21. In a diaper wetness monitoring system, a transmitter arrangement comprising:
a transmitter housing having first and second portions that are pivotally joined to one another for straddling an edge of a diaper;
a resilient clamping element for urging the first and second portions of the transmitter housing toward a closed position with the diaper interposed therebetween; and
a transmitter installed within the transmitter housing, said transmitter being electrically coupled to an electrical contact installed on a selectable one of the first and second portions, the electrical contact being arranged to communicate electrically with a corresponding electrical contact of the diaper in response to said resilient clamping element.

22. The transmitter arrangement of claim 21, wherein said resilient clamping means comprises a magnetic element.

23. The transmitter arrangement of claim 21, wherein said transmitter is installed within the first portion of said transmitter housing, and the electrical contact is disposed on the exterior of the first portion of said transmitter housing so as to be intermediate of the first and second portions when in the closed position, there being further provided an electrical interconnection arrangement between said contact and said transmitter.

24. The transmitter arrangement of claim 21, wherein said transmitter is encased in a waterproof containment arrangement that encases the first and second portions of said transmitter housing and extends therebetween to form a hinge that joins the first and second portions of said transmitter housing pivotally to one another.

25. The transmitter arrangement of claim 24, wherein the waterproof containment arrangement is devoid of crevices that can harbor bacterial growth.

26. The transmitter arrangement of claim 24, wherein the waterproof containment arrangement encases said transmitter housing.

27. The transmitter arrangement of claim 21, wherein said transmitter is arranged to transmit a signal containing data uniquely associated with said transmitter.

28. The transmitter arrangement of claim 27, wherein there is further provided a receiver for receiving the signal containing data uniquely associated with said transmitter.

29. The transmitter arrangement of claim 28, wherein said receiver is configured to learn the data uniquely associated with, and received from, said transmitter.

30. The transmitter arrangement of claim 21, wherein said transmitter has active and quiescent modes of operation, and there is further provided a timer for timing the duration of a mode of said transmitter.

31. A bodily fluid containment product for a living being, the bodily fluid containment product being formed in plural stages of manufacture, the bodily fluid containment product comprising:
an absorptive inner layer for communicating with the bodily fluid;
an impervious outer layer for containing the bodily fluid;
a wetness sensor disposed intermediate of said absorptive inner layer and said impervious outer layer, said absorptive inner layer, said impervious outer layer, and said wetness sensor being combined concurrently during manufacture; and
an electrical contact coupled electrically to said wetness sensor.
